(19) United States
(12) Patent Application Publication
(10) Pub. No.: US 2007/0037840 A1
(43) Pub. Date: Feb. 15, 2007

(54) Tricyclic benzimidazoles

(75) Inventor: Wilm Buhr, Konstanz (DE)

Correspondence Address: NATH & ASSOCIATES PLLC, 112 South West Street, Alexandria, VA, 22314, US

(73) Assignee: Altana Pharma AG, Konstanz (DE)

(21) Appl. No.: 10/582,498

(22) PCT Filed: Dec. 16, 2004
(86) PCT No.: PCT/EP04/53544

§ 371 Date: Jun. 9, 2006

(30) Foreign Application Priority Data
Dec. 16, 2003 (EP) 03028846.8

(51) Int. Cl. A61K 31/4745; C07D 471/02

(52) U.S. Cl. 514/292; 546/82

(57) ABSTRACT

The invention relates to 6,7,8,9-Tetrahydro-3H-imidazo[4,5-h]quinolines of formula 1, 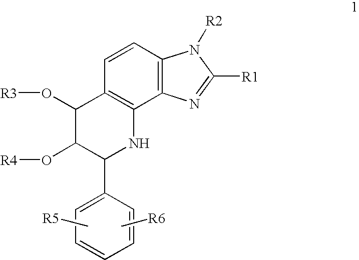 in which the substituents and symbols have the meanings indicated in the description. The compounds have gastric secretion inhibiting and excellent gastric and intestinal protective action properties.

### TECHNICAL FIELD

The invention relates to novel compounds, which are used in the pharmaceutical industry as active compounds for the production of medicaments.

### PRIOR ART

In the international patent applications WO 04/087701 (ALTANA Pharma AG) and WO 04/054984 (ALTANA Pharma AG) substituted benzimidazole derivatives are disclosed which have gastric secretion inhibiting and excellent gastric and intestinal protective action properties.

In the international patent application WO 97/47603 (which corresponds to the U.S. Pat. No. 6,465,505) and in the US patent application U.S. Pat. No. 5,039,806, benzimidazole derivatives having a very specific substitution pattern are disclosed, which are said to be suitable for inhibition of gastric acid secretion and thus can be used in the prevention and treatment of gastrointestinal inflammatory diseases.

In the European patent application EP 0266326 (which corresponds to U.S. Pat. No. 5,106,862) benzimidazole derivatives having a very broad variety of substituents are disclosed, which are said to be active as anti-ulcer agents.

In the European patent application EP 0307078 (which corresponds to the U.S. Pat. No. 5,051,508) substituted quinoline derivatives are disclosed which can be used in therapy as inhibitors of gastric acid secretion.

In the European patent application EP 0307078 (which corresponds to the U.S. Pat. No. 5,051,508) substituted, condensed cinnoline derivatives are disclosed which can be used in therapy as inhibitors of gastric acid secretion.

The German patent application DE 4003587 (which corresponds to the U.S. Pat. No. 5167695) discloses 3H-imidazo[4,5-H](Oxazolo[5,4-H])chinolines, which compounds can be used for the combat of undesired growth of plants.

### SUMMARY OF THE INVENTION

The invention relates to compounds of the formula 1 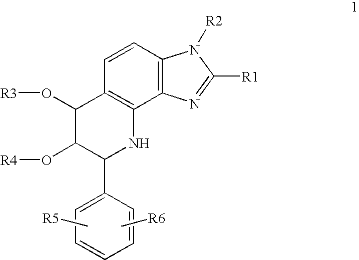
in which
R1 is hydrogen, halogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, fluoro-1-4C-alkoxy-1-4C-alkyl or hydroxy-1-4C-alkyl,
R2 is hydrogen, 1-4C-alkyl, 3-7C-cycloalkyl, 3-7C-cycloalkyl-1-4C-alkyl, 1-4C-alkoxy-1-4C-alkyl, 2-4C-alkenyl, 2-4C-alkynyl, fluoro-1-4C-alkyl or hydroxy-1-4C-alkyl,
R3 is hydrogen, 1-4C-alkyl, 3-7C-cycloalkyl, 3-7C-cycloalkyl-1-4C-alkyl, 1-4C-alkoxy-1-4C-alkyl, fluoro-1-4C-alkyl, fluoro-1-4C-alkoxy-1-4C-alkyl, hydroxy-1-4C-alkyl or a radical aryl-1-4C-alkyl wherein
   aryl is a phenyl substituted by R31 and R32
   where
   R31 is hydrogen, 1-4C-alkyl, fluoro-1-4C-alkyl, 1-4C-alkoxy or fluoro-1-4C-alkoxy and
   R32 is hydrogen, 1-4C-alkyl, fluoro-1-4C-alkyl, 1-4C-alkoxy or fluoro-1-4C-alkoxy,
R4 is hydrogen, 1-4C-alkyl, 3-7C-cycloalkyl, 3-7C-cycloalkyl-1-4C-alkyl, 1-4C-alkoxy-1-4C-alkyl, fluoro-1-4C-alkyl, fluoro-1-4C-alkoxy-1-4C-alkyl or hydroxy-1-4C-alkyl,
   or where R3 and R4 together form a methylene (—CH₂—), an ethylene (—CH₂—CH₂—), a propylene (—CH₂₋CH₂—CH₂—) or a isopropylidene (—C(CH₃)₃—) radical,
R5 is hydrogen, halogen, 1-4C-alkyl or fluoro-1-4C-alkyl
R6 is hydrogen, halogen, 1-4C-alkyl or fluoro-1-4C-alkyl
   and the salts of these compounds.

Halogen within the meaning of the invention is bromo, chloro and fluoro.

1-4C-Alkyl represents a straight-chain or branched alkyl group having 1 to 4 carbon atoms. Examples which may be mentioned are the butyl, isobutyl, sec-butyl, tert-butyl, propyl, isopropyl, ethyl and the methyl group.

3-7C-Cycloalkyl represents cyclopropyl, cyclobutyl, cyclopentyl, cyclohexyl and cycloheptyl, of which cyclo-propyl, cyclobutyl and cyclopentyl are preferred.

3-7C-Cycloalkyl-1-4C-alkyl represents one of the aforementioned 1-4C-alkyl groups, which is substituted by one of the aforementioned 3-7C-cycloalkyl groups. Examples which may be mentioned are the cyclopropylmethyl, the cyclohexylmethyl and the cyclohexylethyl group.

1-4C-Alkoxy represents a group, which in addition to the oxygen atom contains one of the aforementioned 1-4C-alkyl groups. Examples which may be mentioned are the butoxy, isobutoxy, sec-butoxy, tert-butoxy, propoxy, isopropoxy and preferably the ethoxy and methoxy group.

1-4C-Alkoxy-1-4C-alkyl represents one of the aforementioned 1-4C-alkyl groups, which is substituted by one of the aforementioned 1-4C-alkoxy groups. Examples which may be mentioned are the methoxymethyl, the methoxyethyl group and the butoxyethyl group.

1-4C-Alkoxycarbonyl (1-4C-alkoxy-CO—) represents a carbonyl group, to which one of the aforementioned 1-4C-alkoxy groups is bonded. Examples which may be mentioned are the methoxycarbonyl (CH₃O—C(O)—) and the ethoxycarbonyl group (CH₃CH₂O—C(O)—).

2-4C-Alkenyl represents a straight-chain or branched alkenyl group having 2 to 4 carbon atoms. Examples which may be mentioned are the 2-butenyl, 3-butenyl, 1-propenyl and the 2-propenyl group (allyl group).

2-4C-Alkynyl represents a straight-chain or branched alkynyl group having 2 to 4 carbon atoms. Examples which may be mentioned are the 2-butynyl, 3-butynyl, and preferably the 2-propynyl, group (propargyl group).

Fluoro-1-4C-alkyl represents one of the aforementioned 1-4C-alkyl groups, which is substituted by one or more fluorine atoms. Examples which may be mentioned are the fluoromethyl, the difluoromethyl, the trifluoromethyl, the 2-fluoroethyl, the 2,2-difluoroethyl, the 1,2,2-trifluoroethyl, the 2,2,2-trifluoroethyl, the 1,1,2,2-tetrafluoroethyl or the perfluoroethyl radical.

Fluoro-1-4C-alkoxy-1-4C-alkyl denotes one of the abovementioned 1-4C-alkyl radicals which is substituted by a fluoro-1-4C-alkoxy radical. Here, fluoro-1-4C-alkoxy denotes one of the abovementioned 14C-alkoxy radicals which is fully or predominantly substituted by fluorine. Examples of fully or predominantly fluorine-substituted 1-4C-alkoxy which may be mentioned are the 1,1,1,3,3,3-hexafluoro-2-propoxy, the 2-trifluoro-methyl-2-propoxy, the 1,1,1-trifluoro-2-propoxy, the perfluoro-tert-butoxy, the 2,2,3,3,4,4,4-heptafluoro-1-butoxy, the 4,4,4trifluoro-1-butoxy, the 2,2,3,3,3-pentafluoropropoxy, the perfluoroethoxy, the 1,2,2-trifluoroethoxy, in particular the 1,1,2,2-tetrafluoroethoxy, the 2,2,2-trifluoroethoxy, the trifluoromethoxy and preferably the difluoromethoxy radicals. Examples of fluoro-1-4C-alkoxy-1-4C-alkyl radicals which may be mentioned are 1,1,2,2-tetrafluoroethoxymethyl, the 2,2,2-trifluoroethoxymethyl, the trifluoromethoxymethyl, the 1,1,2,2-tetrafluoroethoxyethyl, the 2,2,2-trifluoroethoxyethyl, the trifluoromethoxyethyl and preferably the difluoromethoxymethyl and the difluoromethoxyethyl radical.

Hydroxy-1-4C-alkyl represents one of the aforementioned 1-4C-alkyl groups, which is substituted by a hydroxy group. Examples which may be mentioned are the hydroxymethyl, the 2-hydroxyethyl and the 3-hydroxypropyl group.

Aryl-1-4C-alkyl represents one of the aforementioned 1-4-C-alkyl groups, which is substituted by a aryl radical. Preferred aryl-1-4C-alkyl groups are aryl-CH₂— (substituted benzyl) radicals. Examples of aryl-1-4C-alkyl radicals which are to be mentioned are the p-methylphenyl-CH₂—, the p-trifluoromethylphenyl-CH₂— and especially the p-difluoromethoxyphenyl-CH₂— and the phenyl-CH₂— (benzyl) radical.

Possible salts of compounds of the formula 1—depending on substitution—are especially all acid addition salts. Particular mention may be made of the pharmacologically tolerable salts of the inorganic and organic acids customarily used in pharmacy. Those suitable are water-soluble and water-insoluble acid addition salts with acids such as, for example, hydrochloric acid, hydrobromic acid, phosphoric acid, nitric acid, sulfuric acid, acetic acid, citric acid, D-gluconic acid, benzoic acid, 2-(4-hydroxybenzoyl)benzoic acid, butyric acid, sulfosalicylic acid, maleic acid, lauric acid, malic acid, fumaric acid, succinic acid, oxalic acid, tartaric acid, embonic acid, stearic acid, toluenesulfonic acid, methanesulfonic acid or 3-hydroxy-2-naphthoic acid, where the acids are used in salt preparation—depending on whether a mono- or polybasic acid is concerned and on which salt is desired—in an equimolar quantitative ratio or one differing therefrom.

Pharmacologically intolerable salts, which can initially be obtained, for example, as process products in the production of the compounds according to the invention on the industrial scale, are converted into the pharmacologically tolerable salts by processes known to the person skilled in the art.

The compounds of the formula 1 have at least three centers of chirality in the skeleton. The invention thus provides all feasible stereoisomers in any mixing ratio, including the pure stereoisomers, which are a preferred subject matter of the invention.

It is known to the person skilled in the art that the compounds according to invention and their salts, if, for example, they are isolated in crystalline form, can contain various amounts of solvents. The invention therefore also comprises all solvates and in particular all hydrates of the compounds of the formula 1, and also all solvates and in particular all hydrates of the salts of the compounds of the formula 1.

One special embodiment (embodiment a) of the invention relates to compounds of the formula 1, in which R3 is a radical aryl-1-4C-alkyl
wherein
aryl is a phenyl substituted by R31 and R32
where
R31 is hydrogen, 1-4C-alkyl, fluoro-1-4C-alkyl, 1-4C-alkoxy or fluoro-1-4C-alkoxy and
R32 is hydrogen, 1-4C-alkyl, fluoro-1-4C-alkyl, 1-4C-alkoxy or fluoro-1-4C-alkoxy,

And wherein R1, R2, R4, R5 and R6 have the meanings as indicated in the outset.

Another special embodiment of the invention relates to compounds of the formula 1, in which R3 and R4 together form a methylene (—CH₂—), an ethylene (—CH₂—CH₂—), a propylene (—CH₂—CH₂—CH₂—) or a isopropylidene (—C(CH₃)₃—) radical,

And wherein R1, R2, R5 and R6 have the meanings as indicated in the outset.

Compounds which are to be mentioned, are those compounds of the formula 1, where
R1 is hydrogen, halogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, fluoro-1-4C-alkoxy-1-4C-alkyl or hydroxy-1-4C-alkyl,
R2 is hydrogen, 1-4C-alkyl, 3-7C-cycloalkyl, 3-7C-cycloalkyl-1-4C-alkyl, 1-4C-alkoxy-1-4C-alkyl, 2-4C-alkenyl, 2-4C-alkynyl, fluoro-1-4C-alkyl or hydroxy-1-4C-alkyl,
R3 is hydrogen, 1-4C-alkyl, 3-7C-cycloalkyl, 3-7C-cycloalkyl-1-4C-alkyl, 1-4C-alkoxy-1-4C-alkyl, fluoro-1-4C-alkyl, fluoro-1-4C-alkoxy-1-4C-alkyl or hydroxy-1-4C-alkyl,
R4 is hydrogen, 1-4C-alkyl, 3-7C-cycloalkyl, 3-7C-cycloalkyl-1-4C-alkyl, 1-4C-alkoxy-1-4C-alkyl, fluoro-1-4C-alkyl, fluoro-1-4C-alkoxy-1-4C-alkyl or hydroxy-1-4C-alkyl
R5 is hydrogen, halogen, 1-4C-alkyl or fluoro-1-4C-alkyl
R6 is hydrogen, halogen, 1-4C-alkyl or fluoro-1-4C-alkyl
and the salts of these compounds.

Compounds which are also to be mentioned, are those compounds of the formula 1, where
R1 is hydrogen, 1-4C-alkyl or 3-7C-cycloalkyl,
R2 is hydrogen, 1-4C-alkyl or 3-7C-cycloalkyl,
R3 is hydrogen, 1-4C-alkyl, 3-5C-cycloalkyl, 1-4C-alkoxy-1-4C-alkyl, fluoro-1-4C-alkoxy-1-4C-alkyl, hydroxy-1-4C-alkyl or a radical aryl-1-4C-alkyl
   wherein
   aryl is a phenyl substituted by R31 and R32
   where
   R31 is hydrogen, 1-4C-alkyl, fluoro-1-4C-alkyl, 1-4C-alkoxy or fluoro-1-4C-alkoxy and
   R32 is hydrogen, 1-4C-alkyl, fluoro-1-4C-alkyl, 1-4C-alkoxy or fluoro-1-4C-alkoxy,
R4 is hydrogen, 1-4C-alkyl or hydroxy-1-4C-alkyl
or where R3 and R4 together form a methylene (—CH₂—), an ethylene (—CH₂—CH₂—), a propylene (—CH₂₋CH₂—CH₂—) or a isopropylidene (—C(CH₃)₃—) radical,
R5 is hydrogen, fluoro, 1-4C-alkyl or fluoro-1-4C-alkyl
R6 is hydrogen, fluoro, 1-4C-alkyl or fluoro-1-4C-alkyl
and the salts of these compounds.

Compounds to be particularly mentioned are those of the formula 1 where
R1 is hydrogen, 1-4C-alkyl or 3-7C-cycloalkyl,
R2 is hydrogen or 1-4C-alkyl,
R3 is hydrogen, 1-4C-alkyl, 3-5C-cycloalkyl, 1-4C-alkoxy-1-4C-alkyl, fluoro-1-4C-alkyl, fluoro-1-4C-alkoxy-1-4C-alkyl or hydroxy-1-4C-alkyl and
R4 is hydrogen, 1-4C-alkyl or hydroxy-1-4C-alkyl
R5 is hydrogen, fluoro, 1-4C-alkyl or fluoro-1-4C-alkyl
R6 is hydrogen, fluoro, 1-4C-alkyl or fluoro-1-4C-alkyl
and the salts of these compounds.

Compounds to be particularly emphasized are those of the formula 1, where
R1 is 1-4C-alkyl,
R2 is hydrogen or 1-4C-alkyl,
R3 is hydrogen, 1-4C-alkyl, 3-7C-cycloalkyl-1-4C-alkyl, 1-4C-alkoxy-1-4C-alkyl, fluoro-1-4C-alkyl, or a radical Aryl-1-4C-alkyl,
   wherein
   Aryl is phenyl,
R4 is hydrogen,
or where R3 and R4 together form an ethylene (—CH₂—CH₂—) radical,
R5 is hydrogen and
R6 is hydrogen,
and the salts of these compounds.

Compounds to be also particularly emphasized are those of the formula 1 where
R1 is 1-4C-alkyl,
R2 is 1-4C-alkyl,
R3 is hydrogen, 1-4C-alkyl or 1-4C-alkoxy-1-4C-alkyl
R4 is hydrogen,
R5 is hydrogen,
R6 is hydrogen
and the salts of these compounds.

Among the compounds of the formula 1 according to the invention, emphasis is given to the optically pure compounds of the formula 1 a 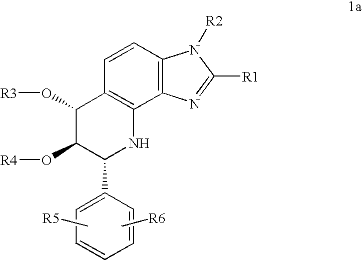
and the salts of these compounds.

Compounds which are to be mentioned, are those compounds of the formula 1a, where
R1 is hydrogen, halogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, fluoro-1-4C-alkoxy-1-4C-alkyl or hydroxy-1-4C-alkyl,
R2 is hydrogen, 1-4C-alkyl, 3-7C-cycloalkyl, 3-7C-cycloalkyl-1-4C-alkyl, 1-4C-alkoxy-1-4C-alkyl, 2-4C-alkenyl, 2-4C-alkynyl, fluoro-1-4C-alkyl or hydroxy-1-4C-alkyl,
R3 is hydrogen, 1-4C-alkyl, 3-7C-cycloalkyl, 3-7C-cycloalkyl-1-4C-alkyl, 1-4C-alkoxy-1-4C-alkyl, fluoro-1-4C-alkyl, fluoro-1-4C-alkoxy-1-4C-alkyl or hydroxy-1-4C-alkyl,
R4 is hydrogen, 1-4C-alkyl, 3-7C-cycloalkyl, 3-7C-cycloalkyl-1-4C-alkyl, 1-4C-alkoxy-1-4C-alkyl, fluoro-1-4C-alkyl, fluoro-1-4C-alkoxy-1-4C-alkyl or hydroxy-1-4C-alkyl
R5 is hydrogen, halogen, 1-4C-alkyl or fluoro-1-4C-alkyl
R6 is hydrogen, halogen, 1-4C-alkyl or fluoro-1-4C-alkyl
and the salts of these compounds.

Compounds which are also to be mentioned, are those compounds of the formula 1 a, where
R1 is hydrogen, 1-4C-alkyl or 3-7C-cycloalkyl,
R2 is hydrogen, 1-4C-alkyl or 3-7C-cycloalkyl,
R3 is hydrogen, 1-4C-alkyl, 3-5C-cycloalkyl, 1-4C-alkoxy-1-4C-alkyl, fluoro-1-4C-alkoxy-1-4C-alkyl, hydroxy-1-4C-alkyl or a radical aryl-1-4C-alkyl
   wherein
   aryl is a phenyl substituted by R31 and R32
   where
   R31 is hydrogen, 1-4C-alkyl, fluoro-1-4C-alkyl, 1-4C-alkoxy or fluoro-1-4C-alkoxy and
   R32 is hydrogen, 1-4C-alkyl, fluoro-1-4C-alkyl, 1-4C-alkoxy or fluoro-1-4C-alkoxy,
R4 is hydrogen, 1-4C-alkyl or hydroxy-1-4C-alkyl
or where R3 and R4 together form a methylene (—CH₂—), an ethylene (—CH₂—CH₂—), a propylene (—CH₂—CH₂—CH₂—) or a isopropylidene (—C(CH₃)₃—) radical,
R5 is hydrogen, fluoro, 1-4C-alkyl or fluoro-1-4C-alkyl
R6 is hydrogen, fluoro, 1-4C-alkyl or fluoro-1-4C-alkyl
and the salts of these compounds.

Compounds to be particularly mentioned are those of the formula 1a where
R1 is hydrogen, 1-4C-alkyl or 3-7C-cycloalkyl,
R2 is hydrogen or 1-4C-alkyl,
R3 is hydrogen, 1-4C-alkyl, 3-5C-cycloalkyl, 1-4C-alkoxy-1-4C-alkyl, fluoro-1-4C-alkoxy-1-4C-alkyl or hydroxy-1-4C-alkyl and
R4 is hydrogen, 1-4C-alkyl or hydroxy-1-4C-alkyl
R5 is hydrogen, fluoro, 1-4C-alkyl or fluoro-1-4C-alkyl
R6 is hydrogen, fluoro, 1-4C-alkyl or fluoro-1-4C-alkyl
and the salts of these compounds.

Compounds to be particularly emphasized are those of the formula 1a, where
R1 is 1-4C-alkyl,
R2 is hydrogen or 1-4C-alkyl,
R3 is hydrogen, 1-4C-alkyl, 3-7C-cycloalkyl-1-4C-alkyl, 1-4C-alkoxy-1-4C-alkyl, fluoro-1-4C-alkyl, or a radical Aryl-1-4C-alkyl,
   wherein
   Aryl is phenyl,
R4 is hydrogen,
or where R3 and R4 together form an ethylene (—CH₂—CH₂—) radical,
R5 is hydrogen and
R6 is hydrogen,
and the salts of these compounds.

Compounds to be also particularly emphasized are those of the formula 1a where
R1 is 1-4C-alkyl,
R2 is 1-4C-alkyl,
R3 is hydrogen, 1-4C-alkyl or 1-4C-alkoxy-1-4C-alkyl
R4 is hydrogen,
R5 is hydrogen,
R6 is hydrogen
and the salts of these compounds.

Particularly preferred are the compounds given as final products of formula 1 in the examples, and the salts of these compounds.

The compounds according to the invention can be synthesized from corresponding starting compounds, for example according to the reaction schemes given below. The synthesis is carried out in a manner known to the expert, for example as described in more detail in the following examples.

The compounds of the formula 1 can be obtained for example starting from compounds of the formula 2 following the reaction sequence shown in scheme 1. Oxidation of compounds of the formula 2 to compounds of the formula 3 is performed by standard procedures, for example using manganese dioxide. Reduction of the keto group in compounds of the formula 3 to the corresponding diols of the formula 1 (R3, R4=H) can be carried out, for example, using sodium borohydride followed, if desired, by customary derivatization reactions which are familiar to the person skilled in the art (e.g. by alkylation or by acylation) to give compounds of the formula 1 with R3 and/or R4? H.

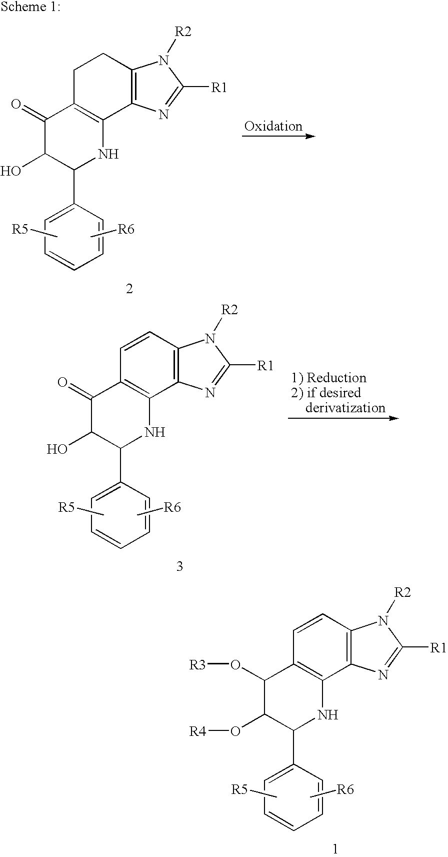

Compounds of the formula 2 can be prepared for example as outlined in scheme 2. In a first step ketones of the formula 4 are reacted with protected phenylisoserine derivatives of the formula 5 (wherein Y is a suitable leaving group, for example an ethoxy group and Prot is a suitable protecting group like a suitable silyl radical, for example a ^{t}BuMe2Si-radical) to give compounds of the formula 6 and/or compounds of the formula 2. Compounds of the formula 6, if obtained, can be deprotected by standard procedures to the desired compounds of the formula 2.

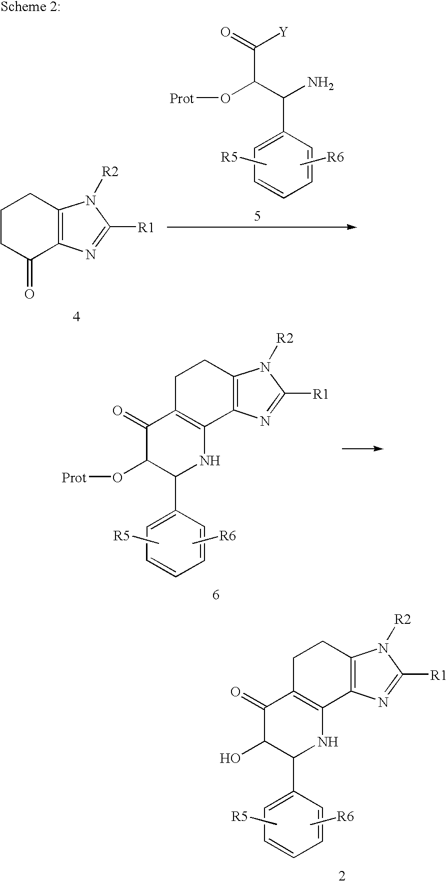

The synthesis as outlined in scheme 2a leads to the preferred optically pure compound of the formula 1a by reacting ketones of the formula 4 with optically pure phenylisoserin derivatives of the formula 5a and further chemical transformations as described for scheme 1.

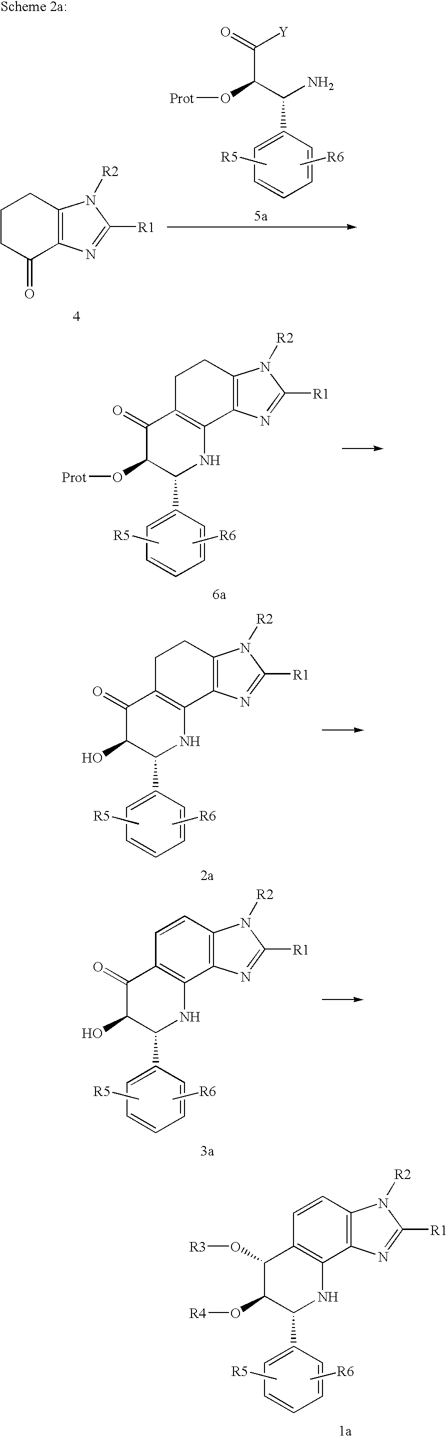

Ketones of the formula 4 are known, for example from Helvetica Chimica Acta (1979), 62, 507, or can be prepared in a manner as shown for example in scheme 3 (route A). 3-Nitro-2-aminophenol can be reacted in a first step with a suitable benzyl derivative, for example benzylchloride, and the amino group of the reaction product of the formula 8 (known from J. Heterocyclic Chem. (1983), 20, 1525) is converted to the di-amide of the formula 9. Subsequent reduction under standard conditions, for example using hydrazine N₂H₄ in the presence of FeCl₃, leads to the formation of the primary amide of the formula 10, whose amine functionality can be alkylated in a next step, for example under reductive alkylation conditions, to compounds of the formula 11. The following cyclization step is performed under standard conditions, for example under acidic conditions using POCl₃, to give compounds of the formula 12 whose hydrogenation to the desired compounds of the formula 4 is performed in manner known to the expert, for example as described by H. Oelschlaeger and H. Giebenhain in Archiv der Pharmazie, 1973, 306, 485-489.

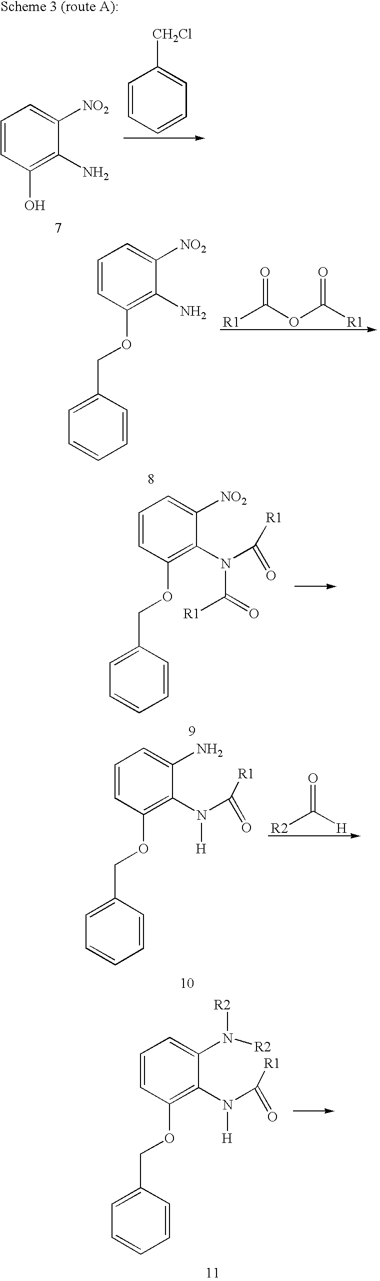

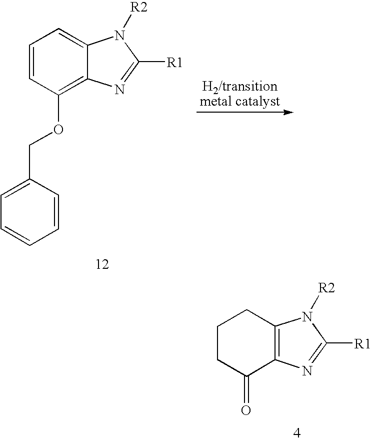

Alternatively, the ketones of the formula 4 can be prepared from con:pounds of the formula 15 by a cyclization reaction in the presence of a primary amine as shown in scheme 4 (route B). Compounds of the formula 15 are known, for example from H. Stetter and K. Hoehne, Chem. Ber., 1958, 91,1123-1128, or can be prepared in an analogous manner starting from 2-nitroresorcin as shown in scheme 4.

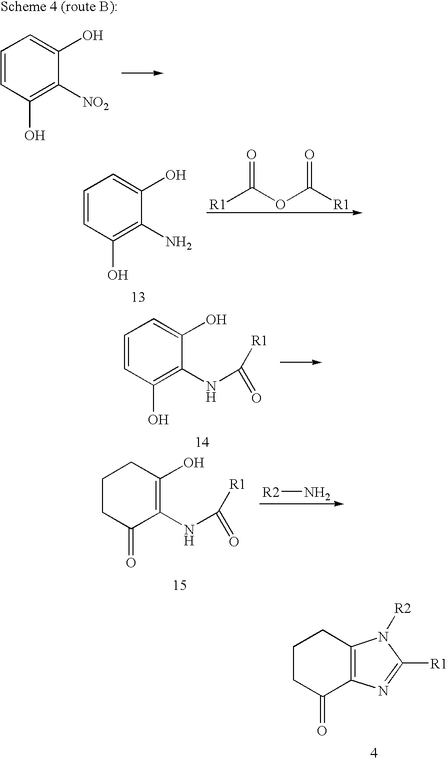

Phenylisoserine derivatives of the formula 5 or 5a can be prepared in analogy to methods known in literature (see for example J. Amer. Chem. Soc. (1998), 120, 431) or by methods known to the expert, for example by reaction under basic conditions of the corresponding unprotected phenylisoserine derivatives of the formula 16 with suitable protection group precursor Prot-X with a suitable leaving group X, like a suitable silyl chloride, for example ^{t}BuMe2SiCl, as shown in Scheme 5.

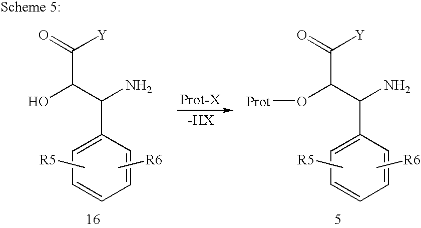

Compounds of the formula 16 are known or can be prepared by methods known to the expert, for example by epoxidaton of the corresponding cinnamic acid derivatives of the formula 17, followed by a ring opening reaction or directly by a aminohydroxylation reaction. Both variants can be performed in a stereoselective way, which leads for example to compounds of the formula 16a, as shown in Scheme 6.

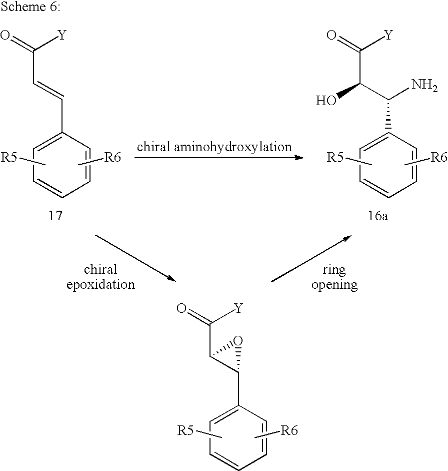

Another synthesis for compounds of the formula 1 is shown in scheme 7 by way of example for the preferred compounds of the formula 1a.

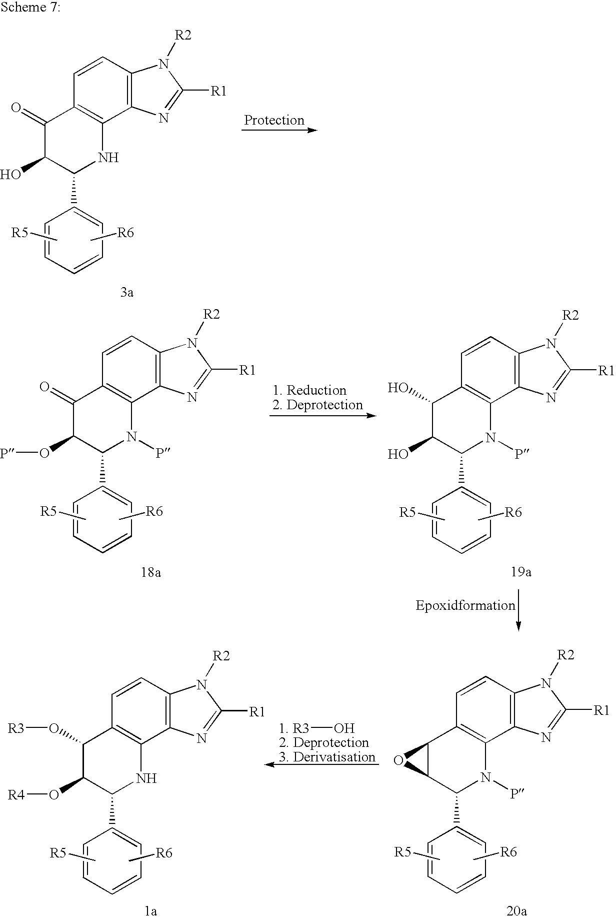

Protection of the hydroxyl group and of the amino group of compounds of formula 3a provides compounds of formula 18a and is performed by standard procedures and standard protection groups (P′ and P″), like for example formyl, acetyl, pivaloyl or benzoyl. Reduction of the keto group in compounds of formula 18a by simultaneous or subsequent deprotection of the hydroxyl group leads to the corresponding diols of the formula 19a and can be carried out by methods known to a person skilled in the art, for example, using sodium borohydride followed by treatment with potassium carbonate. Epoxid formation to yield epoxide compounds of the formula 20a is carried out, for example under Mitsunobu conditions or by other reaction conditions known to the expert. Stereoselective epoxid opening by using alcohols of the general formula R3-OH under acidic catalysis, followed, if desired, by subsequent standard derivatisation reactions, like for example, esterification or further alkylation by simultaneous or subsequent deprotection of the amino functionality leads the desired compounds of the formula 1a.

Still another synthesis for compounds of the formula 1 is shown in scheme 8 by way of example for the preferred compounds of the formula 1a.

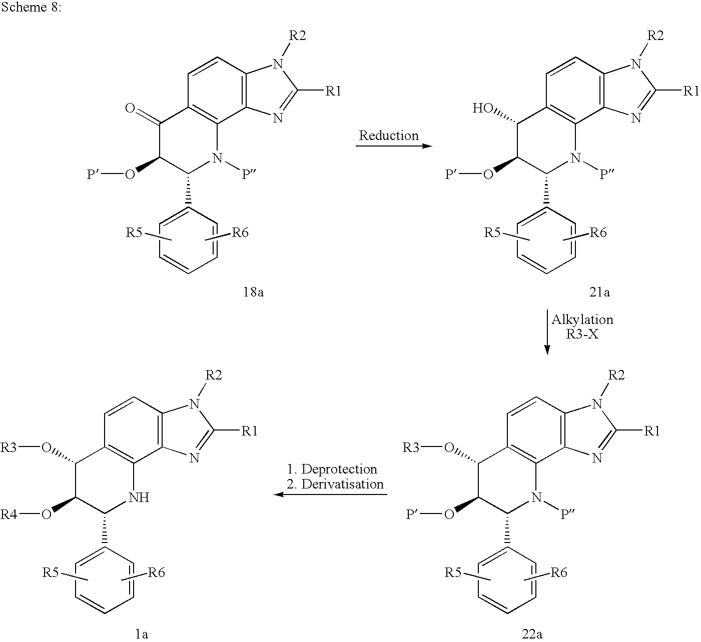

In this reaction sequence, which can lead, as shown in Scheme 8, in a stereoselective way to the preferred compounds of the formula 1a, the starting compounds of the general formula 18a is selectively reduced under standard conditions, for(-example using sodium borohydride, to give compounds of formula 21a which are transformed by alkylation with a suitable alkylation reagent R3-X, wherein X is a suitable leaving group, like for example triflate, to compounds of formula 22a. After deprotection of the reaction products of the formula 2a by methods known to the person skilled in the art, compounds of the formula 1a wherein R4 is hydrogen are obtained. The final compounds of formula 1a with R3 and/or R4 unequal hydrogen are obtained by further derivatization reactions which are known to the expert.

The compounds of formula 1, where R3 and R4 together form a methylen (—CH2-), an ethylen (—CH2-CH2-), a propylen (—CH2-CH2-CH2-) or a isopropyliden (—C(CH₃)₃—) radical, are designated as compounds of the formula 1* and can be prepared for example by following the reaction sequence shown in scheme 9 (for n=0, 1, 2).

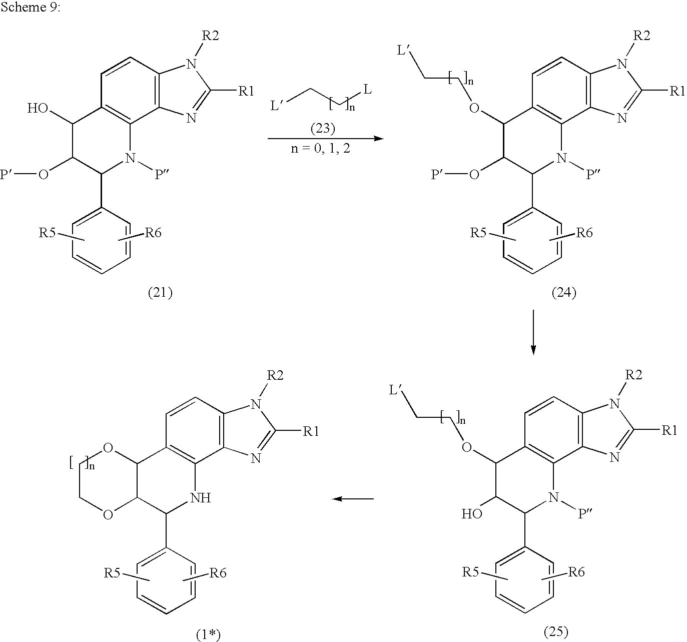

Compounds of the formula 21 are reacted with a compound of the formula 23 to which two leaving groups L and L′ are attached, like for example L=triflate radical and L′=halogen atom, like for example a fluorine atom. The resulting compounds of the formula 24 can be converted to compounds of the formula 25 by methods known to the expert, and the final cyclization reaction to compounds of the formula 1* is likewise carried out in a known manner known per se, for example after reaction with a base, like for example sodium hydride and is carried out before or together with the deprotection of the amino functionality.

The invention further relates to the processes and the process intermediates described in the above schemes, in particular the processes described in scheme 1, scheme 7, scheme 8 and scheme 9 and the process intermediates of the general formulae 3 and 3a as outlined in schemes 1 and 2a.

The following examples serve to illustrate the invention in greater detail without restricting it. Likewise, further compounds of the formula 1 whose preparation is not described explicitly can be prepared in an analogous manner or in a manner familiar per se to the person skilled in the art using customary process techniques. The abbreviation min stands for minute(s), h for hour(s) and m.p. for melting point.

### EXAMPLES

I. Final Products of Formula 1

### 1. (6R,7R,8R)-2,3-Dimethyl-7-hydroxy-6-(2-methoxyethoxy)-8-phenyl-6,7,8,9-tetrahydro-3H-imidazo[4,5h]quinoline

To a at −10° C. cooled stirred suspension of 2.50 g (8.08 mmol) (6R,7R,8R)-2,3-dimethyl-6,7-dihydroxy-8-phenyl-6,7,8,9-tetrahydro-3H-imidazo[4,5-h]quinoline in 2-methoxyethanol was added 0.99 ml (17.8 mmol) conc. sulphuric acid. The reaction mixture was stirred for further 5 h. The mixture was poured out into a saturated sodium hydrogen carbonate solution and extracted with ethyl acetate three times. The combined organic layers were concentrated in vacuo and purified by column chromatography (dichloromethane/methanol: 100/3 and ethyl acetate: 100) to give 0.40 g (1.09 mmol/13%) of the title product as a light brown foam.

¹H-NMR (200 MHz, CDCl₃): δ=2.51 (s, 3 H), 3.38 (s, 3 H), 3.54-3.63 (m, 2 H), 3.66 (s, 3 H), 3.86-4.11 (m, 2 H), 4.21 (dd, 1 H), 4.49 (d, 1 H), 4.87 (dd, 1 H), 6.67 (d, 1 H), 7.23 (1d, 1 H), 7.31-7.42 (m, 3 H), 7.52-7.56 (m,2 H).

### 2. (6S,7R,8R)-2,3-Dimethyl-7-hydroxy-6-(2-methoxyethoxy)-8-phenyl-6,7,8,9-tetrahydro-3H-imidazo[4,5-]quinoline

To a at −10° C. cooled stirred suspension of 2.50 g (8.08 mmol) (6R,7R,8R)-2,3-dimethyl-6,7-dihydroxy-8-phenyl-6,7,8,9tetrahydro-3H-imidazo[4,5-h]quinoline in 2-methoxyethanol was added 0.99 ml (17.8 mmol) conc. sulphuric acid. The reaction mixture was stirred for further 5 h. The mixture was poured out into a saturated sodium hydrogen carbonate solution and extracted with ethyl acetate three times. The combined organic layers are concentrated in vacuo and purified by column chromatography (dichloromethane/methanol: 100/3 and ethyl acetate: 100) to give 1.50 g (4.09 mmol /51%) of the title product as a light brown foam.

¹H-NMR (200 MHz, CDCl₃): δ=2.52 (s, 3 H), 3.40 (s, 3 H), 3.58-3.63 (m, 2 H), 3.66 (s, 3 H), 3.78-4.00 (m, 2 H), 4.06 (bd, 1H), 4.55-4.59 (m, 2 H), 6.61 (d, 1 H), 7.09 (1d, 1 H), 7.30-7.40 (m, 3 H), 7.50-7.54 (m,2H).

### 3. (6R,7R,8R)-2,3-Dimethyl-6-ethoxy-7-hydroxy-8-phenyl-6,7,8,9-tetrehydro-3H-imidazo[4,5h]-quinoline

To a at −10° C. cooled stirred suspension of 2.00 g (6.50 mmol) (6R,7R,8R)-2,3-dimethyl-6,7-dihydroxy-8-phenyl-6,7,8,9-tetrahydro-3H-imidazo[4,5-h]quinoline in ethanol (40 ml) was added 0.79 ml (14.3 mmol) conc. sulphuric acid. The reaction was warmed up to 25° C. and was stirred for further 3 h. The mixture was poured out into a saturated sodium hydrogen carbonate solution and extracted with ethyl acetate three times. The combined organic layers were concentrated in vacuo and purified by column chromatography (dichloromethane/methanol: 100/3). The obtained solid was crystallized from ethyl acetate to give 0.09 g (0.27 mmol/4.0%) of the title product as a colourless solid with a melting point of 177.5° C. (ethyl acetate).

### 4. (6S,7R,8R)-2,3-Dimethyl-6-ethoxy-7-hydroxy-8-phenyl-6,7,8,9-tetrahydro-3H-imidazo[4,5-h]-quinoline

To a at −10° C. cooled stirred suspension of 2.00 g (6.50 mmol) (6R,7R,8R)-2,3-dimethyl-6,7-dihydroxy-8-phenyl-6,7,8,9-tetrahydro-3H-imidazo[4,5-h]quinoline in ethanol (40 ml) was added 0.79 ml (14.3 mmol) conc. sulphuric acid. The reaction was warmed up to 25° C. and stirred for further 3 h. The mixture was poured out into a saturated sodium hydrogen carbonate solution and extracted with ethyl acetate three times. The combined organic layers were concentrated in vacuo and purified by column chromatography (dichloromethane/methanol: 100/3). The obtained solid was crystallized from ethyl acetate to give 1.50 g (4.44 mmol/68%) of the title product as a colourless solid with a melting point of 169.9° C. (ethyl acetate).

### 5. (6R,7R,8R)-2,3-Dimethyl-6,7-dihydroxyl-8-phenyl-6,7,8,9-tetrahydro-3H-imidazo[4,5-h]quinoline

To a stirred suspension of 2.00 g (6.50 mmol) (7R,8R)-7-hydroxy-2,3-dimethyl-8-phenyl-8,9-dihydro-3H,7H-imidazo[4,5-h]quinolin-6-one in methanol (40 ml) was added 0.50 g (13.22 mmol) sodium boron hydride and it was stirred for further 1 h. Subsequently the reaction was quenched by pouring it out into a saturated ammonium chloride solution. The mixture was extracted with dichloromethane three times. The combined organic layers were concentrated in vacuo and purified by column chromatography (dichloromethane/methanol: 100/3 to 13/1). The obtained solid was crystallized from acetone to give 2.00 g (6.50 mmol/100%) of the diastereomeric mixture of the expected diols. This mixture was separated by column chromatography (ethyl acetate) to give 1.75 g (5.65 mmol/87%) of the title product as a colourless solid with a melting point of 224.7° C. (ethyl acetate).

### 6. (6S,7R,8R)-2,3-Dimethyl-6,7-dihydroxy-8-phenyl-6,7,8,9tetrahydro-3H-imidazo[4,5-h]quinoline

To a stirred suspension of 2.00 g (6.50 mmol) (7R,8R)-7-hydroxy-2,3-dimethyl-8-phenyl-8,9-dihydro-3H,7H-imidazo[4,5-h]quinolin-6-one in methanol (40 ml) was added 0.50 g (13.22 mmol) sodium boron hydride and it was stirred for further 1 h. Subsequently the reaction was quenched by pouring it out into a saturated ammonium chloride solution. The mixture was extracted with dichloromethane three times. The combined organic layers were concentrated in vacuo and purified by column chromatography (dichloromethane/methanol: 100/3 to 13/1). The obtained solid was crystallized from acetone to give 2.00 g (6.50 mmol/100%) of the diastereomeric mixture of the expected diols. This mixture is separated by column chromatography (ethyl acetate) to give 0.15 g (0.48 mmol/7.5%) of the title product as a colourless solid with a melting point of 235.4° C. (ethyl acetate).

### 7. (6R,7R,8R)-2,3-Dimethyl-7-hydroxy-6-(2,2-difluoroethoxy)-8-phenyl-6,7,8,9-tetrahydro-3H-imidazo[4,5-h]quinoline

A suspension of 1.30 g (3.12 mmol) (6R,7R,8R)-9-acetyl-2,3-dimethyl-7-hydroxy-6-(2,2-difluoroethoxy)-8-phenyl-6,7,8,9-tetrahydro-3H-imidazo[4,5-h]quinoline and 1.73 g (12.5 mmol) potassium carbonate in 2-aminoethanol (20.0 ml) was stirred at 80° C. for 41 h. Subsequently the reaction was quenched by pouring out into a saturated ammonium chloride solution. The product was filtered off, washed with water and purified by column chromatography (dichloromethane/methanol: 98/2) to give 0.35 g (0.94 mmol/30%) of the title product.

¹H-NMR (200 MHz, CDCl₃): δ=2.52 (s, 3 H), 3.67 (s, 3 H), 3.81-3.99 (m, 2 H), 4.22 (t, 1 H), 4.46 (d, 1 H), 4.90 (d, 1 H), 5.83 (tt, 1 H), 6.71 (d, 1 H), 7.23 (d, 1 H), 7.33-7.42 (m, 3 ), 7.50-7.54 (m, 2 H).

### 8. (6R,7R,8R)-Benzyloxy-2,3-dimethyl-7-hydroxy-8-phenyl-6,7,8,9-tetrahydro-3H-imidazo[4,5-h]quinoline

A suspension of 2.30 g (5.20 mmol) (6R,7R,8R)-9-acetyl-6-benzyloxy-2,3-dimethyl-7-hydroxy-8-phenyl-6,7,8,9-tetrahydro-3H-imidazo[4,5-h]quinoline and 7.20 g (52.0 mmol) potassium carbonate in 2-amino-ethanol (30.0 ml) was stirred at 100° C. for 3 h. Subsequently the reaction was quenched by pouring out into a saturated ammonium chloride solution. The product was filtered off, washed with water and purified by column chromatography (dichloromethane/methanol: 98/2) to give 1.35 g (3.38 mmol/65%) of the title product as a colourless solid with a melting point of 179° C. (dichloromethane/methanol).

### 9. (6R,7R,8R)-6-Cycloproylmethoxy-2,3-dimethyl-7-hydroxy-8-phenyl-6,7,8,9-tetrehydro-3H-imidazo[4,5h]quinoline

A suspension of 1.90 g (4.70 mmol) (6R,7R,8R)-9-acetyl-6-cycloproylmethoxy-2,3-dimethyl-7-hydroxy-8-phenyl-6,7,8,9-tetrahydro-3H-imidazo[4,5-h]quinoline and 6.50 g (47.0 mmol) potassium carbonate in 2-aminoethanol (30.0 ml) was stirred at 100° C. for 3 h. Subsequently the reaction was quenched by pouring out into a saturated ammonium chloride solution. The product was filtered off, washed with water and purified by column chromatography (dichloromethane/methanol: 98/2) to give 1.28 g (3.25 mmol/75%) of the title product as a colourless solid with a melting point of 173° C. (dichloromethane/methanol).

### 10. (6R,7R,8R)-7-Hydroxy-2-methyl-6-(2-methoxyethoxy)-8-phenyl-6,7,8,9-tetrahydro-3H-imidazo[4,6h]quinoline

To a at −10° C. cooled stirred suspension of 1.60 g (8.08 mmol) (6R,7R,8R)-2-methyl-6,7-dihydroxy-8-phenyl-6,7,8,9-tetrahydro-3H-imidazo[4,5-h]quinoline in 2-methoxyethanol (32 ml) is added 0.66 ml (11.9 mmol) conc. sulphuric acid and the reaction was stirred for further 4.5 h. The mixture was poured out into a saturated sodium hydrogen carbonate solution and extracted with dichloromethane three times. The combined organic layers were concentrated in vacuo and purified by column chromatography (dichloromethane/methanol: 13/1 and ethyl acetate: 100) to give 0.20 g (0.57 mmol/10%) of the title product as a light brown foam.

¹H-NMR (200 MHz, CDCl₃): δ=2.48 (s, 3 H), 3.36 (s, 3 H), 3.53-3.68 (m, 2 H), 3.87-3.89 (m, 1 H), 4.03-4.16 (m,1 H), 4.21 (t, 1 H), 4.47 (d, 1 H), 4.85 (d, 1 H), 6.75 (d, 1 H), 7.18 (1d, 1 H), 7.22-7.42 (m, 3 H), 7.44-7.58 (m,2 H).

### 11. (6S,7R,8R-7-Hydroxy-2-methyl-6-(2-methoxyethoxy)-8-phenyl-6,7,8,9-tetrahydro-3H-imidazo[4,5-h]quinoline

To a at −10° C. cooled stirred suspension of 1.60 g (8.08 mmol) (6R,7R,8R)-2-methyl-6,7-dihydroxy-8-phenyl-6,7,8,9-tetrahydro-3H-imidazo[4,5-h]quinoline in 2-methoxyethanol (32.0 ml) was added 0.66 ml (11.9 mmol) conc. sulphuric acid and the reaction was stirred for further 4.5 h. The mixture was poured out into a saturated sodium hydrogen carbonate solution and extracted with dichloromethane three times. The combined organic layers were concentrated in vacuo and purified by column chromatography (dichloromethane/methanol: 13/1 and ethyl acetate: 100) to give 0.98 g (2.77 mmol/51%) of the title product as a light brown foam.

¹H-NMR (200 MHz, CDCl₃): δ=2.45 (s, 3 H), 3.39 (s, 3 H), 3.58-3.63 (m, 2 H), 3.74-4.18 (m, 3 H), 4.51 (mc, 2 H), 6.68 (d, 1 H), 7.02 (1d, 1 H), 7.26-7.37 (m, 3 H), 7.40-7.51 (m,2 H).

### 12. (5R,6R,10R)-16,17-Dimethyl-6-phenyl-2,3,6,10,17-hexahydro-5H-1,4-dioxa-7,15,17-triaza-cyclopenta[a]phonanthrene

To a at −4° C. cooled suspension of 2.00 g (4.60 mmol) (6R,7R,8R)-9-acetyl-2,3-dimethyl-6-hydroxy-8-phenyl-7-pivaloyloxy-6,7,8,9-tetrahydro-3H,-imidazo[4,5-h]quinoline in THF (20 ml) was added 9.20 ml (9.20 mmol) 2-fluoroethyl triflate (1M in dichloromethane) and 9.30 ml (9.30 mmol) bis-(trimethylsilyl)-sodium amide (1 M in THF). This mixture was stirred for 1 h at −40° C. Subsequently the reaction was quenched by pouring out into saturated ammonium chloride solution and it was extracted with dichloromethane three times. The combined organic layers were concentrated in vacuo and purified by column chromatography (dichloromethane/methanol: 100/3) to give 1.95 g of a crude product that was transformed with any further purification by stirring with 2.12 g (15.4 mmol) potassium carbonate in 2-aminoethanol (30.0 ml at 80° C. for 28 h. Subsequently the reaction mixture was quenched by pouring out into a saturated ammonium chloride solution. The product was filtered off, washed with water and purified by column chromatography (dichloromethane/methanol: 98/2) to give 0.65 g (2.00 mmol/43%) of the title product as a light yellow solid with a melting point of 248-250° C.

### 13. (6R,7R,8R)-6,7-Dihydroxy-2-methyl-8-phenyl-6,7,8,9tetrahydro-3H-imidazo[4,5h]quinoline

To a stirred suspension of 2.34 g (7.90 mmol) (7R,8R)-7-hydroxy-2-methyl-8-phenyl-8,9-dihydro-3H,7H-imidazo[4,5-h]quinolin-6-one in methanol (45 ml) was added 0.40 g (10.0 mmol) sodium boron hydride and it was stirred for further 1 h. Subsequently the reaction was quenched by pouring it out into a saturated ammonium chloride solution. The mixture was extracted with dichloromethane ten times. The combined organic layers were concentrated in vacuo and purified by column chromatography (dichloromethane/methanol: 13/1) to give 1.80 g (6.01 mmol/77%) of the title product.

¹H-NMR (200 MHz, CDCl₃): δ=2.39 (s, 3 H), 3.60-3.71 (m, 1 H), 4.28 (d, 1 H), 4.65 (t, 1 H), 6.75 (d, 1 H), 7.11 (d, 1 H), 7.30-7.48 (m, 5 H).

II. Starting Compounds and Intermediates:

### A. 2-Benzyloxy-6-nitroaniline

To a solution of 50.0 g (0.31 mol) 2-amino-3-nitrophenol in ethanol (400 ml) were added 43.5 ml (0.38 mol) benzyl chloride, 47.8 g (0.35 mol) potassium carbonate and 2.00 g (13.3 mmol) sodium iodide and it was stirred at 80° C. for 3.5 h. Subsequently the mixture was concentrated in vacuo, redissolved in dichloromethane, washed with water, dried over sodium sulfate, filtrated over sand and concentrated in vacuo again. The crude product was purified by column chromatography (cyclohexane/ethyl acetate: 8/2) to give 76.0 g (0.31 mol/96%) of the title product.

¹H-NMR (200 MHz, CDCl₃): δ=5.11 (s, 2 H), 6.57 (t, 1 H), 6.95 (d,1 H), 7.35-7.44 (m, 5 H), 7.73 (d, 1 H).

### B. N-Acetyl-2-benzyloxy-6-nitro-acetanilide

To a stirred reaction mixture of 76.0 g (0.31 mol) 2-benzyloxy-6-nitroaniline in acetic anhydride (469 ml) were added 7.60 ml (0.12 mol) methane sulfonic acid and the mixture was stirred for 2 h at 120° C. Afterwards the acetic anhydride was removed in vacuo and the residue was poured into ice water. This mixture was neutralised with concentrated ammonia solution and extracted with dichloromethane three times. The combined organic layers were concentrated and dried in vacuo to give 99.9 g (0.30 mol/98%) of the title product with a melting point of 113.8° C. (dichloromethane).

### C. 2-Amino-6-benzyloxy-acetanilide

To a stirred mixture of 99.6 g (0.30 mol) N-acetyl-2-benzyloxy-6-nitro-acetanilide, activated carbon (59.7 g) and 30.0 g (18.5 mmol) iron (III) chloride in methanol (2.60 I) at 70° C. were added dropewise 147 ml (3.03 mol) hydrazine hydrate and the mixture was stirred for further 5 h. Subsequently the mixture was filtrated over kieselgur and concentrated in vacuo. The crude mixture was suspended in a saturated ammonium chloride solution and extracted with dichloromethane twice. The combined organic layers were concentrated in vacuo and the crude product was reslurred in diethyl ether to give 50.5 g (0.20 mol/65%) of the title product with a melting point of 146.9° C. (diethyl ether).

### D. 4-Benzyloxy-1,2-dimethyl-1H-benzimidazole

To a stirred mixture of 4.00 g (17.0 mmol) 2-amino-6-benzyloxy-acetanilide in dichloromethane (8.0 ml) were added 4.00 ml (4.30 mmol) phoshoryl chloride and the mixture was stirred at 70° C. for 5 h. Subsequently the mixture was poured into ice water, neutralised by adding sodium hydroxide solution (6 N) and extracted with dichloromethane three times. The combined organic layers were concentrated in vacuo and the crude product was purified by column chromatography (diethyl ether/petrol ether: 7/3) to give 3.09 g (12.2 mmol/72%) of the title product with a melting point of 130.9° C. (diethyl ether/petrol ether).

### E. 1,2-Dimethyl-1,5,6,7-tetrahydro-benzoimidazol-4-one

Route A: A suspension of 2.00 g (7.93 mmol) 4-benzyloxy-1,2-dimethyl-1H-benzimidazole and 1.70 g palladium on carbon (10%) in methanol (50 ml) was stirred in an autoclave at a hydrogen pressure of 150 bar at 70° C. for 20 h. Afterwards the catalyst was filtered off and the methanol was removed in vacuo. The crude product was purified by column chromatography (dichloromethane/methanol: 100/3 to 13/1) to give 0.14 g (0.85 mmol/11%) of the title product with a melting point of 98.1° C. (dichloromethane/methanol).

Route B: To a stirred mixture of 29.0 g (0.17 mol) 2-acetylamino-3-hydroxy-cyclohex-2-enone in xylene (580 ml) were added acetic acid (57 ml) and dropewise 116 ml (0.23 mol) methylamine (2 M in THF). The reaction mixture was heated to 155° C. for 5 h, cooled down to 25° C. and stirred for further 20 h. Afterwards the mixture was concentrated in vacuo and the crude product was purified by column chromatography (ethyl acetate/methanol: 8/2) to yield 21.4 g (0.13 mol/77%) of the title product with a melting point of 98.1° C. (ethyl acetate/methanol).

### F. (2R,3R)-3-amino-2-tert.-butyl-dimethyl-silanyloxy)-3-phenyl propionic acid ethyl ester

1323 g (4.06 mole) of (R, R)-phenylisoserine ethyl ester were dissolved in 6.6. L of dichloromethane. To this solution, 397.4 g of imidazole and 724 g of t-butyldimethylsilyl chloride were added. The mixture was stirred for 16 hrs at RT. The reaction mixture was washed subsequently with 6 L and 4 L of water. The resulting clear dichloromethane layer was dried over sodium sulphate, filtered and concentrated under reduced pressure. The obtained 1509 g of the title compound were used as such without further purification for the next reaction steps.

### G. (7R,8R)-7-Hydroxy-2,3-dimethyl-8-phenyl-5,7,8,9-tetrehydro-3H,4H-imidazo[4,5-h]quinolin-6-one

A mixture of 6.20 g (37.8 mmol) 1,2-dimethyl-1,5,6,7-tetrahydro-benzoimidazol-4-one and 12.5 g (38.6 mmol) (2R,3R)-3-amino-2-(tert.-butyl-dimethyl-silanyloxy)-3-phenyl propionic acid ethyl ester was heated to 170° C. and was stirred for 5.5 h. Afterwards the solid was purified by column chromatography (dichloromethane/methanol: 100/1 to 13/1) to give 6.35 g (20.5 mmol/54%) of the title product as a light brown solid with a melting point of 262.3° C. (dichloromethane/methanol).

### H. (7R,8R)-7-tert-Butyl-dimethyl-silanyl-oxy)-2,3-dimethyl-8-phenyl-5,7,8,9-tetrahydro-3H,4H-imidazo[4,5-h]quinolin-6-one

A mixture of 6.20 g (37.8 mmol) 1,2-dimethyl-1,5,6,7-tetrahydro-benzoimidazol-4-one and 12.5 g (38.6 mmol) (2R,3R)-3-amino-2-(tert.-butyl-dimethyl-silanyloxy)-3-phenyl propionic acid ethyl ester was heated to 170° C. and was stirred for 5.5 h. Afterwards the solid was purified by column chromatography (dichloromethane/methanol: 100/1 to 13/1) to give 2.20 g (5.19 mmol/14%) of the title product. This compound was transformed by acetic standard conditions without any characterisation into (7R,8R)-7-hydroxy-2,3-dimethyl-8-phenyl-5,7,8,9-tetrahydro-3H,4H-imidazo [4,5-h]quinolin-6-one.

### I. (7R,8R-7-Hydroxy-2,3-dimethyl-8-phenyl-8,9-dihydro-3H,7H-imidazo[4,5-h]quinolin-6-one

A reaction mixture of 6.20 g (20.0 mmol) (7R,8R)-7-hydroxy-2,3-dimethyl-8-phenyl-5,7,8,9-tetrahydro-3H,4H-imidazo[4,5-h]quinolin-6-one and 19.0 g (197 mmol) manganese dioxide in dichloromethane (250 ml) was stirred for 20 h at 25° C. Afterwards the manganese residues were filtered off by using kieselgur. The crude product was purified by column chromatography (dichloromethane/methanol: 100/1 to 13/1) and crystallized from acetone to give 4.70 g (15.3 mmol/76%) of the title product as a solid with a melting point of 235.1° C. (acetone).

### J. (7R,8R)-2,3-Dimethyl-8-phenyl-7-pivaloyloxy-8,9-dihydro-3H,7H-imidazo[4,5h]quinolin-6-one

To a suspension of 98.6 g (0.32 mol) (7R,8R)-7-hydroxy-2,3-dimethyl-8-phenyl-8,9-dihydro-3H,7H-imidazo[4,5-h]quinolin-6-one in dichloromethane (500 ml) was added 110 ml (0.63 mol) n-ethyl-diisopropylamine and 15.5 g (0.12 mol) 4-dimethylaminopyridine. The mixture was cooled to 0° C., 78.0 ml (0.63 mol) pivaloyl chloride was added dropwise and it was stirred for 18 h at 0-5° C. Subsequently the reaction was quenched by adding methanol (5.0 ml) and water (500 ml). The mixture was extracted with dichloromethane three times. The combined organic layers were concentrated in vacuo and the crude product was reslurried in petrol ether, filtered off and dried in vacuum to give 120 g (0.31 mol/97%) of the title product.

¹H-NMR (200 MHz, CDCl₃): δ=1.08 (s, 9 H), 2.54 (s, 3 H), 3.69 (s, 3 H), 4.91 (d, 1 H), 5.78 (d, 1 H), 6.72 (d, 1 H), 7.31-7.41 (m, 3 H), 7.50-7.57 (m, 2 H), 7.75 (d, 1 H).

### K. (7R,8R)-9-Acetyl-2,3-dimethyl-8-phenyl-7-pivaloyloxy-8,9-dihydro-3H,7H-imidazo[4,5h]quinolin-6-one

To a suspension of 154 g (0.39 mol) (7R,8R)-2,3-dimethyl-8-phenyl-7-pivaloyloxy -8,9-dihydro-3H,7H-imidazo[4,5-h]quinolin-6-one in acetic anhydride (300 ml) was added dropwise 43.7 ml (0.79 mol) concentrated sulphuric acid and it was stirred for further 20 min. Subsequently the reaction mixture was poured in an ice saturated sodium hydrogen carbonate solution and was extracted with ethyl acetate three times. The combined organic layers were concentrated in vacuo. The crude product was redissolved in dichloromethane/methanol (98/2) and filtered over silica gel to give 154 g (0.36 mol/90%) of the title product.

¹H-NMR (200 MHz, CDCl₃): δ=1.23 (s, 9 H), 2.37 (s, 3 H), 2.66 (s, 3 H), 3.71 (s, 3 H), 5.78 (d, 1 H), 6.62 (s, 1 H), 7.03-7.16 (m, 4 H), 7.25-7.29 (m, 2 H), 7.84 (d, 1 H).

### L. (6R,7R,8R)-9Acetyl-2,3-dimethyl-6-hydroxy-8-phenyl-7-pivaloyloxy-6,7,8,9-tetrahydro-3H,-imidazo[4,5h]quinoline

To a at −50° C. cooled suspension of 120 g (0.28 mol) (7R,8R)-9-acetyl-2,3-dimethyl-8-phenyl-7-pivaloyloxy-8,9-dihydro-3H,7H-imidazo[4,5-h]quinolin-6-one in methanol (950 ml) was added portionwise 14.7 g (0.37 mol) sodium borohydride and it was stirred for further 1 h. Subsequently the reaction mixture was acidified to pH 3 by adding ice and hydrochloric acid (2 N). The mixture was neutralized with sodium hydrogen carbonate and was extracted with dichloromethane three times. The combined organic layers were concentrated in vacuo to give 117 g (0.27 mol/97%) of the title product.

¹H-NMR (200 MHz, CDCl₃): δ=1.27 (s, 9 H), 1.97 (s, 3 H), 2.63 (s, 3 H), 3.77 (s, 3 H), 4.80 (d, 1 H), 5.09 (dd, 1 H), 5.87 (d, 1 H), 7.12-7.17 (m, 5 H), 7.31 (d, 1 H), 7.55 (d, 1 H).

### M. (6R,7R,8R)-9-Acetyl-2,3-dimethyl-6,7-dihydroxy-8-phenyl-6,7,8,9-tetrahydro-3H,-Imidazo[4,5-h]-quinoline

A reaction mixture of 2.00 g (4.60 mmol) (6R,7R,8R)-9-acetyl-2,3-dimethyl-6-hydroxy-8-phenyl-7-pivaloyloxy-6,7,8,9-tetrahydro-3H-imidazo[4,5-h]quinoline and 1.90 g (13.8 mmol) potassium carbonate in methanol (20 ml) was stirred for 3 h. Subsequently the reaction mixture was quenched by adding saturated ammonium chloride solution. The mixture was extracted with dichloromethane/methanol (13/1) three times. The combined organic layers were concentrated in vacuo. The crude product was redissolved in dichloromethane/methanol (9/1) and filtered over silica gel to give 1.20 g (3.41 mmol/74%) of the title product.

¹H-NMR (200 MHz, D₆-DMSO): δ=2.04 (s, 3 H), 2.59 (s, 3 H), 3.13-3.24 (m, 1 H), 3.74 (s, 3 H), 4.46 (dd, 1 H), 5.35 (d, 1 H), 7.13-7.46 (m, 7 H).

### N. (6S,7R,8R)-9-Acetyl-2,3-dimethyl-6,7-epoxy-8-phenyl-6,7,8,9-tetrahydro-3H-imidazo[4,5-h]quinoline

To a at 0° C. cooled reaction mixture of 30.0 g (85.3 mmol) (6R,7R,8R)-9-acetyl-2,3-dimethyl-6,7-dihydroxy-8-phenyl-6,7,8,9-tetrahydro-3H-imidazo[4,5-h]quinoline in DMF (150 ml) was added dropwise 29.1 ml (111 mmol) tri-n-butylphosphine and 23.6 g (111 mmol) diisopropyl azodicarboxylate and it was stirred for further 1 h. Subsequently the reaction mixture was quenched by adding ice and saturated ammonium chloride solution. The crude product was filtered off to give 26.3 g (78.9 mmol/92%) of the title product with a melting point of 200-205° C. (water).

### O. (5R,6R,10R)-7-Acetyl-16,17-dimethyl-phenyl-2,3,6,10,17-hexahydro-5H-1,4-dioxa-7,15,17-triaza-cyclopenta[a]phenanthrene

To a at −40° C. cooled suspension of 2.00 g (4.60 mmol) (6R,7R,8R)-9-acetyl-2,3-dimethyl-6-hydroxy-8-phenyl-7-pivaloyloxy-6,7,8,9-tetrahydro-3H,-imidazo[4,5-h]quinoline in THF (20 ml) was added 9.20 ml (9.20 mmol) 2-fluoroethyl triflate (1M in dichloromethane) and 9.30 ml (9.30 mmol) bi(trimethylsilyl)-sodium amide (1 M in THF). This mixture was stirred for 1 h at −40° C. Subsequently the reaction was quenched by pouring out into saturated ammonium chloride solution and it was extracted with dichloromethane three times. The combined organic layers were concentrated in vacuo and purified by column chromatography (dichloromethane/methanol: 100/3) to give 1.95 g of a crude product that was transformed with any further purification by stirring with 2.12 g (15.4 mmol) potassium carbonate in 2-aminoethanol (30.0 ml at 80° C. for 28 h. Subsequently the reaction mixture was quenched by pouring out into a saturated ammonium chloride solution. The product was filtered off, washed with water and purified by column chromatography (dichloromethane/methanol: 98/2) to give 0.50 g (1.32 mmol/29%) of the title product as a light yellow foam.

¹H-NMR (200 MHz, CDCl₃): δ=2.19 (s, 3 H), 2.64 (s, 3 H), 3.31 (q, 1 H), 3.74 (s, 3 H), 3.66-4.11 (m, 4 H), 4.51 (d, 1 H), 5.66 (d, 1 H), 7.12-7.44 (m, 7 H).

### P. 2-Methyl-1,5,6,7-tetrehydrobenzoimidazolone

A mixture of 50.0 g (0.29 mol) 2-acetylamino-3-hydroxy-cyclohex-2-enone and 300 g (3.89 mmol) ammonium acetate in acetic acid (500 ml) was stirred under reflux for 7 h, cooled down to 25° C. and stirred for further 20 h. Afterwards the mixture was concentrated in vacuo, coevaporated with toluene two times. The crude product was purified by column chromatography (dichloromethane/methanol: 100/3) and reslurried from diethyl ether to yield 36.5 g (0.24 mol/82%) of the title product.

¹H-NMR (200 MHz, CDCl₃): δ=2.11-2.24 (m, 2 H), 2.52-2.58 (m, 5 H), 2.82-2.88 (m, 2 H).

### Q. (6R,7R,8R)-9 Acetyl-benzyloxy-2,3-dimethyl-7-hydroxylphenyl-6,7,8,9-tetrahydro-3H-imidazo[4,5-h]quinoline

To a at 0° C. cooled stirred suspension of 2.00 g (6.00 mmol) (6S,7R,8R)-9-acetyl-2,3-dimethyl-6,7-epoxy-8-phenyl-6,7,8,9-tetrahydro-3H-imidazo[4,5-h]quinoline in benzyl alcohol (20.0 ml) was added concentrated phosphoric acid (0.10 ml) and the reaction mixture was stirred for further 20 h at 2° C. Subsequently the mixture was poured out into a saturated hydrogen carbonate solution and was extracted with dichloromethane two times. The combined organic layers were concentrated in vacuo and the crude product was purified by column chromatography (ethyl acetate) to give 2.30 g (5.20 mmol/87%) of the title product.

¹H-NMR (200 MHz, CDCl₃): δ=2.24 (s, 3 H), 2.62 (s, 3 H), 3.69 (s, 3 H), 3.62-3.79 (m, 1 H), 4.56 (q, 1 H), 5.01 (q, 2 H), 5.70 (d, 1 H), 7.04-7.53 (m, 7 H).

### R. (6R,7R,8R)-9-Acetyl-6-cycloproylmethoxy-2,3-dimethyl-7-hydroxy-8-phenyl-6,7,8,9-tetrahydro-3H-imidazo[4,5-h]quinoline

To a at 0° C. cooled stirred suspension of 2.00 g (6.00 mmol) (6S,7R,8R)-9-acetyl-2,3-dimethyl-6,7-epoxy-8-phenyl-6,7,8,9-tetrahydro-3H-imidazo[4,5-h]quinoline in cyclopropyl-methanol (20.0 ml) was added concentrated phosphoric acid (0.10 ml) and the reaction mixture was stirred for further 20 h at 2° C. Subsequently the mixture was poured out into a saturated hydrogen carbonate solution and was extracted with dichloromethane two times. The combined organic layers were concentrated in vacuo and the crude product was purified by column chromatography (ethyl acetate) to give 1.90 g (4.69 mmol/78%) of the title product.

¹H-NMR (200 MHz, CDCl₃): δ=0.33-0.40 (m, 2 H), 0.62-0.71 (m, 2 H), 1.22-1.34 (m, 1 H), 2.23 (s, 3 H), 2.36 (s, 3 H), 3.54-3.63 (m, 1 H), 3.73 (s, 3 H), 3.69-3.86 (m 2 H), 4.40 (d, 1 H), 5.69 (d, 1 H), 7.10-7.45 (m, 7H).

### S. (7R,8R)-7-Hydroxy-2-methyl-8-phenyl-5,7,8,9-tetrahydro-3H,4H-imidazo [4,5h]quinolin-6-one

A mixture of 5.50 g (36.6 mmol) 2-methyl-1,5,6,7-tetrahydro-benzoimidazol-4-one and 14.8 g (45.8 mmol) phenylisoserine in 2-methoxyethanol (100 ml) was stirred for 14 days under reflux. Afterwards the reaction mixture was concentrated in vacuo and purified by column chromatography (dichloromethane/methanol: 100/1 to 13/1). The product fractions were reslurried from acetone to give 4.30 g (14.6 mmol/40%) of the title product as a yellow solid.

¹H-NMR (200 MHz, CDCl₃): δ=2.29 (s, 3 H), 2.57-2.72 (m, 4 H), 3.91 (d, 1 H), 4.43 (d, 1 H), 7.31-7.50 (m, 5 H).

### T. (7R,8R)-7-Hydroxy-2-methyl-8-phenyl-8,9-dihydro-3H,7H-imidazo[4,5h]quinolin-6-one

A reaction mixture of 4.00 g (13.5 mmol) (7R,8R)-7-hydroxy-2-methyl-8-phenyl-5,7,8,9-tetrahydro-3H,4H-imidazo[4,5-h]quinolin-6-one and 20.0 g (207 mmol) manganese dioxide in dichloromethane (80 ml) was stirred for 17 h at 25° C. Afterwards the manganese residues were filtered off by using kieselgur. The crude product was purified by column chromatography (dichloromethane/methanol: 100/1 to 13/1) and crystallized from acetone to give 2.44 g (8.18 mmol/61%) of the title product.

¹H-NMR (200 MHz, CDCl₃): δ=2.46 (s, 3 H), 4.20-4.36 (m, 1 H), 4.57 (d, 1 H), 6.80 (d, 1 H), 7.35-7.47 (m, 6 H).

### U. (6R,7R,8R)-9-Acetyl-2,3-dimethyl-7-hydroxy-6-(2,2-difluoroethoxy)-8-phenyl-6,7,8,9-tetrahydro-3H-imidazo[4,5-h]quinoline

To a at −5° C. cooled suspension of 2.00 g (4.60 mmol) (6R,7R,8R)-9-acetyl-2,3-dimethyl-6-hyroxy-8-phenyl-7-pivaloyloxy-6,7,8,9-tetrahydro-3H,-imidazo[4,5h]quinoline in THF (20 ml) was added 1.08 ml (5.05 mmol) 2,2-difluoroethyl triflate and 0.36 g (5.05 mmol) sodium hydride (60% dispersion in mineral oil). This mixture was stirred for 0.2 h at 0° C. Subsequently the reaction was quenched by pouring out into saturated ammonium chloride solution and was extracted with dichloromethane three times. The combined organic layers were concentrated in vacuo and purified by column chromatography (dichloromethane/methanol: 100/3) to give 2.3 g of a crude product that was transformed with any further purification by stirring with 4.60 g (33.3 mmol) potassium carbonate in 2-aminoethanol (50 ml) at 60° C. for 3.5 h. Subsequently the reaction mixture was quenched by pouring out into a saturated ammonium chloride solution and was extracted with dichloromethane two times The combined organic layers were concentrated in vacuo and purified by column chromatography (dichloromethane/methanol: 95/5) to give 1.45 g (3.49 mmol/79%) of the title product as a light yellow foam.

¹H-NMR (200 MHz, CDCl₃): δ=2.16 (s, 3 H), 2.62 (s, 3 H), 3.47 (q, 1 H), 3.72 (s, 3 H), 3.67-3.84 (m, 2 H), 4.83 (q, 1 H), 5.76 (d, 1 H),5.82 (tt, 1 H), 7.15-7.29 (m, 6 H), 7.51 (d, 1 H).

Commercial Utility

The compounds of the formula 1 and their salts have valuable pharmacological properties which make them commercially utilizable. In particular, they exhibit marked inhibition of gastric acid secretion and an excellent gastric and intestinal protective action in warm-blooded animals, in particular humans. In this connection, the compounds according to the invention are distinguished by a high selectivity of action, an advantageous duration of action, a particularly good enteral activity, the absence of significant side effects and a large therapeutic range.

“Gastric and intestinal protection” in this connection is understood as meaning the prevention and treatment of gastrointestinal diseases, in particular of gastrointestinal inflammatory diseases and lesions (such as, for example, gastric ulcer, peptic ulcer, including peptic ulcer bleeding, duodenal ulcer, gastritis, hyperacidic or medicament-related functional dyspepsia), which can be caused, for example, by microorganisms (e.g. Helicobacter pylori), bacterial toxins, medicaments (e.g. certain antiinflammatories and antirheumatics, such as NSAIDs and COX-inhibitors), chemicals (e.g. ethanol), gastric acid or stress situations. “Gastric and intestinal protection” is understood to include, according to general knowledge, gastroesophageal reflux disease (GERD), the symptoms of which include, but are not limited to, heartburn and/or acid regurgitation.

In their excellent properties, the compounds according to the invention surprisingly prove to be clearly superior to the compounds known from the prior art in various models in which the antiulcerogenic and the an secretory properties are determined. On account of these properties, the compounds of the formula 1 and their pharmacologically acceptable salts are outstandingly suitable for use in human and veterinary medicine, where they are used, in particular, for the treatment and/or prophylaxis of disorders of the stomach and/or intestine.

A further subject of the invention are therefore the compounds according to the invention for use in the treatment and/or prophylaxis of the abovementioned diseases.

The invention likewise includes the use of the compounds according to the invention for the production of medicaments which are employed for the treatment and/or prophylaxis of the abovementioned diseases.

The invention furthermore includes the use of the compounds according to the invention for the treatment and/or prophylaxis of the abovementioned diseases.

A further subject of the invention are medicaments which comprise one or more compounds of the formula 1 and/or their pharmacologically acceptable salts.

The medicaments are prepared by processes which are known per se and familiar to the person skilled in the art. As medicaments, the pharmacologically active compounds according to the invention (=active compounds) are either employed as such, or preferably in combination with suitable pharmaceutical auxiliaries or excipients in the form of tablets, coated tablets, capsules, suppositories, patches (e.g. as TFS), emulsions, suspensions or solutions, the active compound content advantageously being between 0.1 and 95% and it being possible to obtain a pharmaceutical administration form exactly adapted to the active compound and/or to the desired onset and/or duration of action (e.g. a sustained-release form or an enteric form) by means of the appropriate selection of the auxiliaries and excipients.

The auxiliaries and excipients which are suitable for the desired pharmaceutical formulations are known to the person skilled in the art on the basis of his/her expert knowledge. In addition to solvents, gel-forming agents, suppository bases, tablet auxiliaries and other active compound excipients, it is possible to use, for example, antioxidants, dispersants, emulsifiers, antifoams, flavor corrigents, preservatives, solubilizers, colorants or, in particular, permeation promoters and complexing agents (e.g. cyclodextrins).

The active compounds can be administered orally, parenterally or percutaneously.

In general, it has proven advantageous in human medicine to administer the active compound(s) in the case of oral administration in a daily dose of approximately 0.01 to approximately 20, preferably 0.05 to 5, in particular 0.1 to 1.5, mg/kg of body weight, if appropriate in the form of several, preferably 1 to 4, individual doses to achieve the desired result. In the case of a parenteral treatment, similar or (in particular in the case of the intravenous administration of the active compounds), as a rule, lower doses can be used. The establishment of the optimal dose and manner of administration of the active compounds necessary in each case can easily be carried out by any person skilled in the art on the basis of his/her expert knowledge.

If the compounds according to the invention and/or their salts are to be used for the treatment of the above-mentioned diseases, the pharmaceutical preparations can also contain one or more pharmacologically active constituents of other groups of medicaments, for example: tranquillizers (for example from the group of the benzodiazepines, for example diazepam), spasmolytics (for example, bietamiverine or camylofine), anticholinergics (for example, oxyphencyclimine or phencarbamide), local anesthetics, (for example, tetracaine or procaine), and, if appropriate, also enzymes, vitamins or amino acids.

To be emphasized in this connection is in particular the combination of the compounds according to the invention with pharmaceuticals which inhibit acid secretion, such as, for example, H₂ blockers (e.g. cimetidine, ranitidine), H⁺/K⁺ ATPase inhibitors (e.g. omeprazole, pantoprazole), or further with so-called peripheral anti-cholinergic (e.g. pirenzepine, telenzepine) and with gastrin antagonists with the aim of increasing the principal action in an additive or super-additive sense and/or of eliminating or of decreasing the side effects, or further the combination with antibacterially active substances (such as, for example, cephalosporins, tetracyclines, penicillins, macrolides, nitroimidazoles or alternatively bismuth salts) for the control of Helicobacter pylori. Suitable antibacterial co-components which may be mentioned are, for example, mezlocillin, ampicillin, amoxicillin, cefalothin, cefoxitin, cefataxime, imipenem, gentamycin, amikacin, erythromycin, ciprofloxacin, metronidazole, clarithromycin, azithromycin and combinations thereof (for example clarithromycin+metronidazole).

In view of their excellent gastric and intestinal protection action, the compounds of formula 1 are suited for a free or fixed combination with those medicaments (e.g. certain antiinflammatories and antirheumatics, such as NSAIDS), which are known to have a certain ulcerogenic potency. In addition, the compounds of formula 1 are suited for a free or fixed combination with motility-modifying drugs.

Pharmacology

The excellent gastric protective action and the gastric acid secretion-inhibiting action of the compounds according to the invention can be demonstrated in investigations on animal experimental models. The compounds according to the invention investigated in the model mentioned below have been provided with numbers which correspond to the numbers of these compounds in the examples.

Testing of the Secretion-Inhibiting Action on the Perfused Rat Stomach

In Table A which follows, the influence of the compounds of the formula 1 according to the invention on the pentagastrin-stimulated acid secretion of the perfused rat stomach after intraduodenal administration in vivo is shown.

TABLE A

| | Dose | Inhibition of |
|---|---|---|
| | (μmol/kg) | acid secretion |
| No. | i.d. | (%) |
| 1 | 2.0 | >50 |
| 2 | 2.0 | <50 |
| 3 | 2.0 | >50 |
| 4 | 2.0 | <50 |
| 5 | 2.0 | >50 |
| 6 | 2.0 | <50 |
| 7 | 2.0 | >50 |

Methodology

The abdomen of anesthetized rats (CD rat, female, 200-250 g; 1.5 g/kg i.m. urethane) was opened after tracheotomy by a median upper abdominal incision and a PVC catheter was fixed transorally in the esophagus and another via the pylorus such that the ends of the tubes just projected into the gastric lumen. The catheter leading from the pylorus led outward into the right abdominal wall through a side opening.

After thorough rinsing (about 50-100 ml), warm (37° C.) physiological NaCl solution was continuously passed through the stomach (0.5 ml/min, pH 6.8-6.9; Braun-Unita I). The pH (pH meter 632, glass electrode EA 147; φ=5 mm, Metrohm) and, by titration with a freshly prepared 0.01N NaOH solution to pH 7 (Dosimat 665 Metrohm), the secreted HCl were determined in the effluent in each case collected at an interval of 15 minutes.

The gastric secretion was stimulated by continuous infusion of 1 μg/kg (=1.65 ml/h) of i.v. pentagastrin (left femoral vein) about 30 min after the end of the operation (i.e. after determination of 2 preliminary fractions). The substances to be tested were administered intraduodenally in a 2.5 ml/kg liquid volume 60 min after the start of the continuous pentagastrin infusion. The body temperature of the animals was kept at a constant 37.8-38° C. by infrared irradiation and heat pads (automatic, stepless control by means of a rectal temperature sensor).

1. A compound of the formula 1 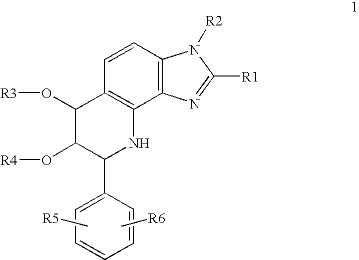
in which
R1 is hydrogen, halogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, fluoro-1-4C-alkoxy-1-4C-alkyl or hydroxy-1-4C-alkyl,
R2 is hydrogen, 1-4C-alkyl, 3-7C-cycloalkyl, 3-7C-cycloalkyl-1-4C-alkyl, 1-4C-alkoxy-1-4C-alkyl, 2-4C-alkenyl, 2-4C-alkynyl, fluoro-1-4C-alkyl or hydroxy-1-4C-alkyl,
R3 is hydrogen, 1-4C-alkyl, 3-7C-cycloalkyl, 3-7C-cycloalkyl-1-4C-alkyl, 1-4C-alkoxy-1-4C-alkyl, fluoro-1-4C-alkyl, fluoro-1-4C-alkoxy-1-4C-alkyl, hydroxy-1-4C-alkyl or a radical aryl-1-4C-alkyl
wherein
aryl is a phenyl substituted by R31 and R32
where
R31 is hydrogen, 1-4C-alkyl, fluoro-1-4C-alkyl, 1-4C-alkoxy or fluoro-1-4C-alkoxy and
R32 is hydrogen, 1-4C-alkyl, fluoro-1-4C-alkyl, 1-4C-alkoxy or fluoro-1-4C-alkoxy,
R4 is hydrogen, 1-4C-alkyl, 3-7C-cycloalkyl, 3-7C-cycloalkyl-1-4C-alkyl, 1-4C-alkoxy-1-4C-alkyl, fluoro-1-4C-alkyl, fluoro-1-4C-alkoxy-1-4C-alkyl or hydroxy-1-4C-alkyl,
or where R3 and R4 together form a methylene (—CH₂—), an ethylene (—CH₂—CH₂—), a propylene (—CH₂—CH₂—CH₂—) or a isopropylidene (—C(CH₃)₃—) radical,
R5 is hydrogen, halogen, 1-4C-alkyl or fluoro-1-4C-alkyl
R6 is hydrogen, halogen, 1-4C-alkyl or fluoro-1-4C-alkyl
or a salt thereof

2. A compound of the formula 1 as claimed in claim 1, in which
in which
R1 is hydrogen, halogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, fluoro-1-4C-alkoxy-1-4C-alkyl or hydroxy-1-4C-alkyl,
R2 is hydrogen, 1-4C-alkyl, 3-7C-cycloalkyl, 3-7C-cycloalkyl-1-4C-alkyl, 1-4C-alkoxy-1-4C-alkyl, 2-4C-alkenyl, 2-4C-alkynyl, fluoro-1-4C-alkyl or hydroxy-1-4C-alkyl,
R3 is hydrogen, 1-4C-alkyl, 3-7C-cycloalkyl, 3-7C-cycloalkyl-1-4C-alkyl, 1-4C-alkoxy-1-4C-alkyl, fluoro-1-4C-alkyl, fluoro-1-4C-alkoxy-1-4C-alkyl or hydroxy-1-4C-alkyl,
R4 is hydrogen, 1-4C-alkyl, 3-7C-cycloalkyl, 3-7C-cycloalkyl-1-4C-alkyl, 1-4C-alkoxy-1-4C-alkyl, fluoro-1-4C-alkyl, fluoro-1-4C-alkoxy-1-4C-alkyl or hydroxy-1-4C-alkyl
R5 is hydrogen, halogen, 1-4C-alkyl or fluoro-1-4C-alkyl
R6 is hydrogen, halogen, 1-4C-alkyl or fluoro-1-4C-alkyl
or a salt thereof.

3. A compound of the formula 1 as claimed in claim 1, in which
R1 is hydrogen, 1-4C-alkyl or 3-7C-cycloalkyl,
R2 is hydrogen, 1-4C-alkyl or 3-7C-cycloalkyl,
R3 is hydrogen, 1-4C-alkyl, 3-5C-cycloalkyl, 1-4C-alkoxy-1-4C-alkyl, fluoro-1-4C-alkyl, fluoro-1-4C-alkoxy-1-4C-alkyl, hydroxy-1-4C-alkyl or a radical aryl-1-4C-alkyl
wherein
aryl is a phenyl substituted by R31 and R32
where
R31 is hydrogen, 1-4C-alkyl, fluoro-1-4C-alkyl, 1-4C-alkoxy or fluoro-1-4C-alkoxy and
R32 is hydrogen, 1-4C-alkyl, fluoro-1-4C-alkyl, 1-4C-alkoxy or fluoro-1-4C-alkoxy,
R4 is hydrogen, 1-4C-alkyl or hydroxy-1-4C-alkyl or where R3 and R4 together form a methylene (—CH₂—), an ethylene (—CH₂—CH₂—), a propylene (—CH₂—CH₂—CH₂—) or a isopropylidene (—C(CH₃)₃—) radical,
R5 is hydrogen, fluoro, 1-4C-alkyl or fluoro-1-4C-alkyl
R6 is hydrogen, fluoro, 1-4C-alkyl or fluoro-1-4C-alkyl
or a salt thereof.

4. A compound of the formula 1 as claimed in claim 1, in which
R1 is 1-4C-alkyl,
R2 is hydrogen or 1-4C-alkyl,
R3 is hydrogen, 1-4C-alkyl, 3-7C-cycloalkyl-1-4C-alkyl, 1-4C-alkoxy-1-4C-alkyl, fluoro-1-4C-alkyl, or a radical Aryl-1-4C-alkyl,
wherein
Aryl is phenyl,
R4 is hydrogen,
or where R3 and R4 together form an ethylene (—CH₂—CH₂—) radical,
R5 is hydrogen and
R6 is hydrogen,
or a salt thereof.

5. A compound of the formula 1 as claimed in claim 1, in which
R1 is 1-4C-alkyl,
R2 is 1-4C-alkyl,
R3 is hydrogen, 1-4C-alkyl or 1-4C-alkoxy-1-4C-alkyl
R4 is hydrogen,
R5 is hydrogen,
R6 is hydrogen
or a salt thereof.

6. A compound of the formula 1 as claimed in claim 1, characterized by the formula 1a, 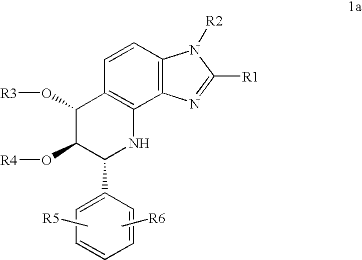
in which
R1 is hydrogen, halogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, fluoro-1-4C-alkoxy-1-4C-alkyl or hydroxy-1-4C-alkyl,
R2 is hydrogen, 1-4C-alkyl, 3-7C-cycloalkyl, 3-7C-cycloalkyl-1-4C-alkyl, 1-4C-alkoxy-1-4C-alkyl, 2-4C-alkenyl, 2-4C-alkynyl, fluoro-1-4C-alkyl or hydroxy-1-4C-alkyl,
R3 is hydrogen, 1-4C-alkyl, 3-7C-cycloalkyl, 3-7C-cycloalkyl-1-4C-alkyl, 1-4C-alkoxy-1-4C-alkyl, fluoro-1-4C-alkyl, fluoro-1-4C-alkoxy-1-4C-alkyl, hydroxy-1-4C-alkyl or a radical aryl-1-4C-alkyl
wherein
aryl is a phenyl substituted by R31 and R32
where
R31 is hydrogen, 1-4C-alkyl, fluoro-1-4C-alkyl, 1-4C-alkoxy or fluoro-1-4C-alkoxy and
R32 is hydrogen, 1-4C-alkyl, fluoro-1-4C-alkyl, 1-4C-alkoxy or fluoro-1-4C-alkoxy,
R4 is hydrogen, 1-4C-alkyl, 3-7C-cycloalkyl, 3-7C-cycloalkyl-1-4C-alkyl, 1-4C-alkoxy-1-4C-alkyl, fluoro-1-4C-alkyl, fluoro-1-4C-alkoxy-1-4C-alkyl or hydroxy-1-4C-alkyl,
or where R3 and R4 together form a methylene (—CH₂—), an ethylene (—CH₂—CH₂—), a propylene (—CH₂—CH₂—CH₂—) or a isopropylidene (—C(CH₃)₃—) radical,
R5 is hydrogen, halogen, 1-4C-alkyl or fluoro-1-4C-alkyl
R6 is hydrogen, halogen, 1-4C-alkyl or fluoro-1-4C-alkyl
or a salt thereof.

7. A compound of the formula 3 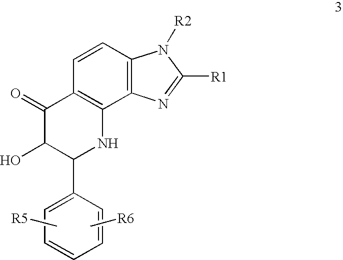
in which
R1 is hydrogen, halogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, fluoro-1-4C-alkoxy-1-4C-alkyl or hydroxy-1-4C-alkyl,
R2 is hydrogen, 1-4C-alkyl, 3-7C-cycloalkyl, 3-7C-cycloalkyl-1-4C-alkyl, 1-4C-alkoxy-1-4C-alkyl, 2-4C-alkenyl, 2-4C-alkynyl, fluoro-1-4C-alkyl or hydroxy-1-4C-alkyl,
R5 is hydrogen, halogen, 1-4C-alkyl or fluoro-1-4C-alkyl
R6 is hydrogen, halogen, 1-4C-alkyl or fluoro-1-4C-alkyl or a salt thereof.

8. A process for the synthesis of a compound of the formula 1 as claimed in claim 1, which comprises reducing a keto group of a compound of formula 3 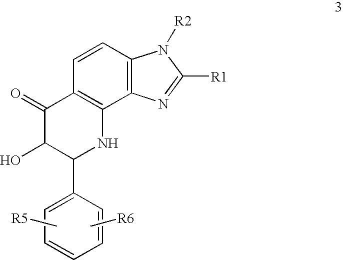
in which
R1 is hydrogen, halogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, fluoro-1-4C-alkoxy-1-4C-alkyl or hydroxy-1-4C-alkyl,
R2 is hydrogen, 1-4C-alkyl, 3-7C-cycloalkyl, 3-7C-cycloalkyl-1-4C-alkyl, 1-4C-alkoxy-1-4C-alkyl, 2-4C-alkenyl, 2-4C-alkynyl, fluoro-1-4C-alkyl or hydroxy-1-4C-alkyl,
R5 is hydrogen, halogen, 1-4C-alkyl or fluoro-1-4C-alkyl
R6 is hydrogen, halogen, 1-4C-alkyl or fluoro-1-4C-alkyl
or a salt thereof,
to obtain a compound of the formula 1 as claimed in claim 1,
and optionally submitting said compound of formula 1 to a derivatization reaction.

9. A pharmaceutical composition comprising a compound as claimed in claim 1 and/or a pharmacologically acceptable salt thereof together with a pharmaceutically acceptable auxiliary and/or excipient.

10. A method of preventing or treating a gastrointestinal disorder in a patient comprising administering to a patient in need thereof, a compound as claimed in claim 1 or a pharmacologically acceptable salt thereof.

11. A process for the synthesis of a compound according to claim 1 of the formula 1a 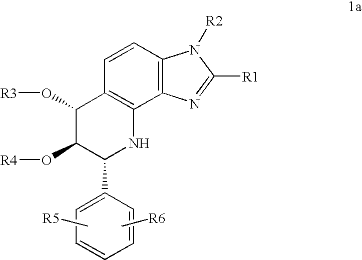
which comprises protecting an amino group and a hydroxyl group of a compound of formula 3a 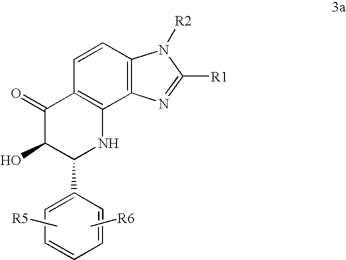
to provide a compound of formula 18a 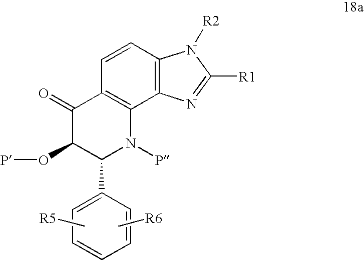
wherein P′ is a protecting group and P″ is a protecting group,
reducing a keto group and deprotecting a hydroxyl group of a compound of formula 18a to obtain a compound of the formula 19a 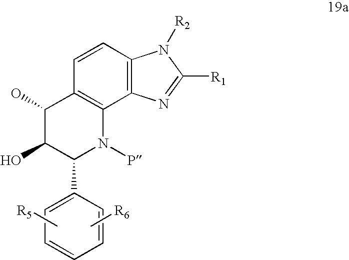
submitting said compound of formula 19a to an epoxidformation reaction to yield a compound of formula 20a 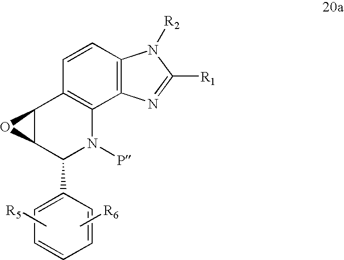
reacting said compound of formula 20a with an alcohol of the formula R3-OH to obtain a compound of formula 1a, and optionally submitting said compound of formula 1a to a derivatization reaction.
